(19) United States
(12) Patent Application Publication
(10) Pub. No.: US 2015/0008192 A1
(43) Pub. Date: Jan. 8, 2015

(54) FLUID DECONTAMINATION METHOD

(71) Applicant: Gregory J. Bruni, Phoenix, AZ (US)

(72) Inventor: Gregory J. Bruni, Phoenix, AZ (US)

(21) Appl. No.: 14/322,184

(22) Filed: Jul. 2, 2014

Related U.S. Application Data
(60) Provisional application No. 61/842,632, filed on Jul. 3, 2013.

(51) Int. Cl. A61L 2/232; A61M 1/36; A61L 2/235; C02F 1/50

(52) U.S. Cl. CPC A61L 2/232; C02F 1/50; A61M 1/3687; A61L 2/235; A61M 2202/0413; A61M 2205/0205; C02F 2303/04
USPC 210/764

(57) ABSTRACT

A method for decontaminating a biologically contaminated fluid, comprising the steps of: providing a substrate comprising an open-cell foam at least substantially coated with a solution consisting essentially of an organo-functional silane-based quarternary ammonium salt at a concentration of between approximately 0.10 percent to approximately 3.7 percent in water; at least substantially drying said solution on said substrate; placing said coated substrate in a container; introducing into said container a fluid to be decontaminated; and agitating the container for a period of time sufficient to substantially biologically decontaminate said fluid.

### CROSS-REFERENCE TO RELATED APPLICATIONS

The present application is related to, and claims the benefit of priority from, U.S. Provisional Application Ser. No. 61/842,632, filed 3 Jul. 2013, the disclosure of which application is incorporated herein by reference in its entirety.

### BACKGROUND

1. Field of the Invention

The present invention relates to fluid decontamination methods, and more particularly to such a method wherein a substrate comprising an open-cell foam at least substantially coated with a solution consisting essentially of an organo-functional silane-based quarternary ammonium salt is placed in contact with a fluid to be decontaminated for a period of time sufficient to substantially decontaminate the fluid.

2. Related Art

Contamination of a fluid by harmful bacteria and other biological contaminants can render that fluid unusable. For example, biological contamination of water can make the water unsuitable for human consumption. Moreover, biological contamination of other liquids, including, for instance, fuels, industrial fluids, blood, or blood plasma may leave those fluids unfit for their intended purpose. Once a fluid is biologically contaminated, it must be decontaminated, or purified, of harmful contaminates before it may be used further.

A number of conventional methods are known for decontaminating a biologically contaminated fluid, including boiling, chemical treatment with chlorine or iodine based chemicals, ultraviolet treatment, or filtration through a purification medium, such as activated carbon, for instance.

Conventional methods suffer from drawbacks, however. For example, boiling can consume large quantities of fuel and may be unsuitable for certain fluids such as gasoline, blood, or blood plasma. Consuming fluids treated with iodine may trigger undesirable allergic reactions in humans. Moreover, chlorine and iodine based chemicals can impart a foul taste to a purified liquid. Additionally, chemical treatments have reduced effectiveness at non-optimal temperatures. Ultraviolet treatment requires equipment that may not be readily available, and suffers from reduced efficacy in fluids with suspended particles. Finally, filtration methods may lose efficacy due to filter media tearing, inadequate sealing, a cracked filter housing, etc.

There thus is a need for an improved fluid decontamination system that does not suffer from the aforementioned drawbacks.

### SUMMARY OF THE INVENTION

The specification discloses a method for biologically decontaminating a fluid, comprising the steps of: Providing a substrate comprising an open-cell foam at least substantially coated with a solution consisting essentially of an organo-functional silane-based quarternary ammonium salt at a concentration of between approximately 0.10 percent to approximately 3.7 percent in water; at least substantially drying said solution on said substrate; placing said coated substrate in a container; introducing into said container a fluid to be decontaminated; and agitating the container for a period of time sufficient to substantially biologically decontaminate said fluid.

The fluid to be biologically decontaminated may, by way of non-limiting example, be selected from the group consisting of biologically contaminated water, fuels, industrial fluids, blood, and blood plasma.

In one embodiment of the invention, the substrate may be an open-cell foam characterized by 25 pores per inch (“ppi”).

In another embodiment, the open-cell foam may be a reticulated polyethylene foam.

In another aspect of the invention, the organo-functional silane-based quarternary ammonium salt may be selected from the group consisting of 3-(trihydroxysilyl) propyldimeythylloctadecyl ammonium chloride, octadecyldimethyl trimethoxysilylpropyl ammonium chloride, and combinations thereof.

Per yet another aspect, the solution includes a suspension of organo-functional silane-based quartenary ammonium salt dissolved in a non-alcoholic solvent including water.

According to a further aspect of the invention, the solution is part of a liquid composition that includes a phenol ethoxylate.

Per a still further aspect of the invention, the solution is part of a liquid composition that includes a phosphate ester.

The concentration of the organo-functional silane-based quarternary ammonium salt may, per one embodiment, be between about 1.0 percent and 1.7 percent, inclusive. Per yet another embodiment, by way of example, the concentration of the organo-functional silane-based quarternary ammonium salt may be between about 1.3 percent and 1.4 percent, inclusive.

Per one embodiment, the step of at least substantially drying the solution on the substrate is accomplished through the evaporation of the water from the solution at room temperature. In other embodiments, the step of drying is carried out under ambient conditions, under the controlled flow of air, under vacuum, under an inert gas (e.g., Nitrogen) environment. Drying may be carried out at any temperature suitable to the drying method; provided that the drying temperature is lower than the melting temperature of the open-cell foam substrate.

The present invention eliminates the issues associated with conventional fluid purification systems. The invention of this disclosure does not require additional energy input by a user to decontaminate a fluid. Additionally, the invention does not chemically alter the fluid being purified. Moreover, the invention is capable of biologically decontaminating both high-clarity and low-clarity fluids. Finally, the invention does not suffer from reduced efficiency due to filter media tearing, inadequate sealing, or from a cracked filter housing.

### BRIEF DESCRIPTION OF THE DRAWINGS

The foregoing and other features and advantages of the present invention will become more readily appreciated when considered in connection with the following detailed description and appended drawings, wherein:

FIG. 1 is a front side view of a representative fluid container;

FIG. 2 is a cross-sectional view taken generally along line 2-2 in FIG. 1;

FIG. 3 is a perspective view of a representative substrate;

FIG. 4A is a chemical formula of a representative organo-functional silane-based molecule useable in the solution of the present invention;

FIG. 4B is stylized view of the representative organo-functional silane-based molecule depicted in FIG. 4A;

FIG. 5 is an enlarged view of the area circumscribed at 5 in FIG. 3 and illustrates, in stylized form, a portion of a surface of the substrate having organo-functional silane-based molecules bonded to it;

FIG. 6A is an enlarged view of the area circumscribed at 5 in FIG. 3 and illustrates, in stylized form, a biological contaminant in proximity to a portion of a surface of the substrate having organo-functional silane-based molecules bonded to it;

FIG. 6B is an enlarged view of the area circumscribed at 3 and illustrates, in stylized form, a cell membrane of a biological contaminant being ruptured by the organo-functional silane-based molecules bonded to the surface of a substrate; and

FIG. 7 is a flow diagram depicting a method of decontaminating a biologically contaminated fluid.

### DETAILED DESCRIPTION

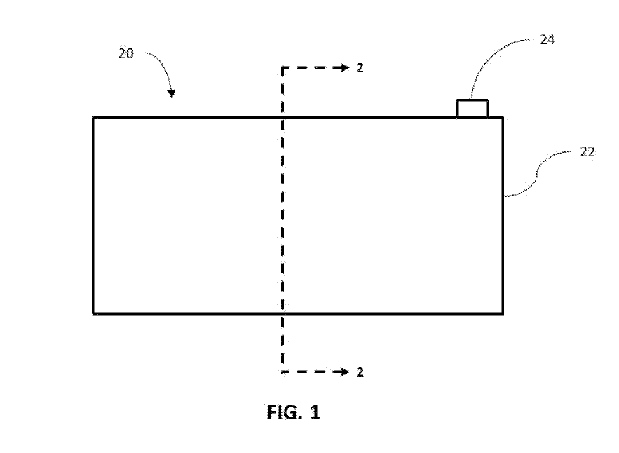

Referring to FIGS. 1 through 7, wherein like numerals indicate like or corresponding parts throughout the several views, the present invention may be seen to generally comprise a method for decontaminating a biologically contaminated fluid, the method comprising the steps of: Providing a substrate comprising an open-cell foam at least substantially coated with a solution consisting essentially of an organo-functional silane-based quarternary ammonium salt at a concentration of between approximately 0.10 percent to approximately 3.7 percent in water; at least substantially drying said solution on said substrate; placing said coated substrate in a container; introducing into said container a fluid to be decontaminated; and agitating the container for a period of time sufficient to substantially biologically decontaminate said fluid.

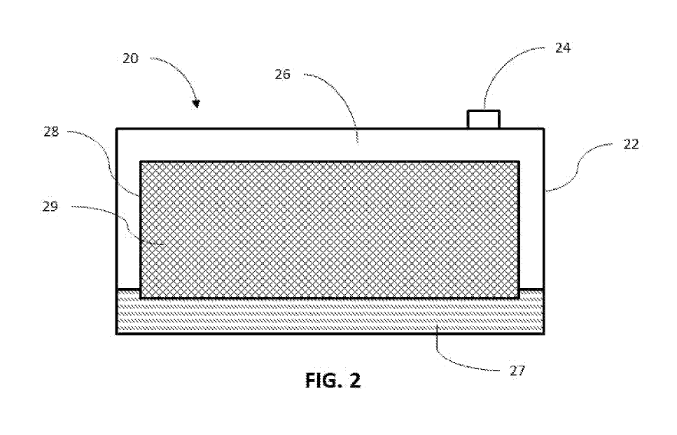

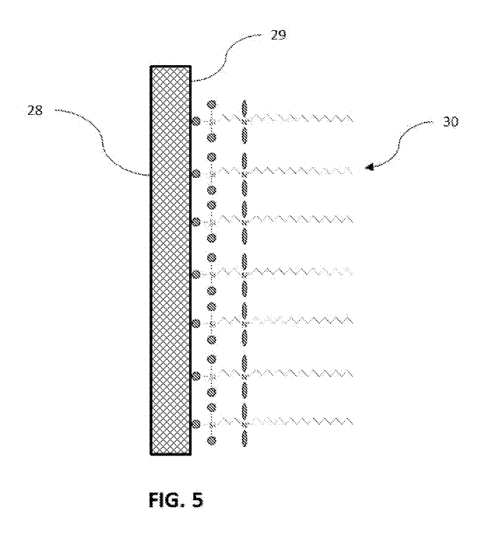

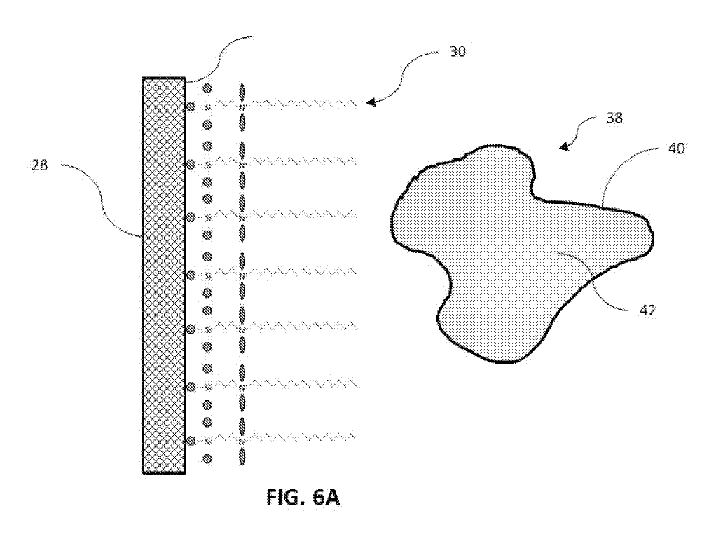

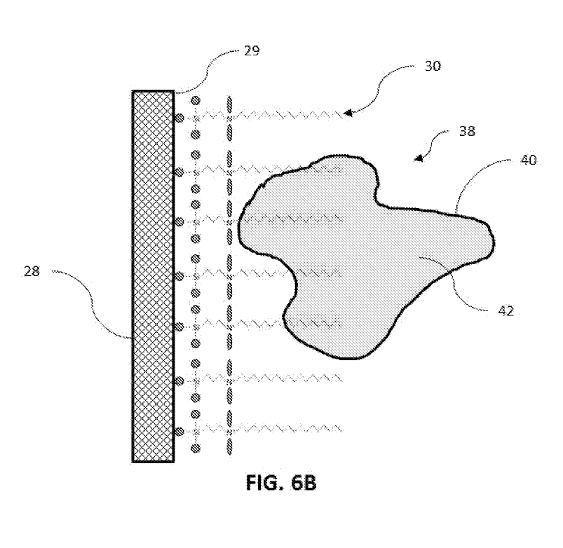

With continuing reference to the drawings, the method of the present invention is, in an exemplary embodiment, carried out in a fluid purification system (indicated generally at 20 in FIG. 1) comprising a container (indicated at 22 in FIG. 1) substantially encasing a substrate (indicated at 28 in FIG. 2). The substrate 28 is treated with a solution consisting essentially of an organo-functional silane-based quarternary ammonium salt at a concentration of between approximately 0.10 percent to approximately 3.7 percent in water. As shown in FIG. 5, the organo-functional silane-based molecules (indicated generally at 30 in FIG. 5) of the solution are characterized in that, when a biological contaminant (indicated at 38 in FIGS. 6A and 6B) contacts the organo-functional silane-based molecules 30, a cell membrane 40 of the biological contaminant 38 is ruptured, resulting in death of the biological contaminant 38.

With specific reference to FIG. 1, container 22 has at least one opening 24 to permit the container 22 to be filled with fluid. The same opening 24 may also permit effluence of fluid from the container 22. But while the exemplary embodiment of FIG. 1 depicts only one opening 24, those skilled in the art will recognize that the container 22 may include multiple openings. For example, the container 22 may include one or more openings both for fluid influx and effluence from the container 22. Indeed, those skilled in the art will appreciate that the container employed in the present invention may take any of numerous forms and sizes, depending upon the amount of fluid being decontaminated, the time required to effect the decontamination, and the area of the substrate employed.

Referring now to FIG. 2, a cross-sectional view of an exemplary fluid decontaminating system 20 for carrying out the methodology of the present invention, taken generally along line 2-2 in FIG. 1, is shown. Per convention, the container 22 includes a cavity 26 capable of holding a volume of a biologically contaminated fluid 27 and the substrate 28. The fluid 27 may be, by way of example and not limitation, drinking water, gasoline, diesel fuel, jet fuel, blood, blood plasma, metal-working fluid, industrial fluid, or any other fluid that may contain undesirable biological contaminants (such as indicated at 38 in FIGS. 6A and 6B).

Referring still to FIG. 2, the fluid purification system 20 further includes a substrate 28 substantially disposed within the cavity 26 of the container 22. The substrate 28 includes at least one surface 29 that may be exposed to the fluid 27, and more particularly to the biological contaminants (indicated at 38 in FIGS. 6A and 6B) in the fluid 27.

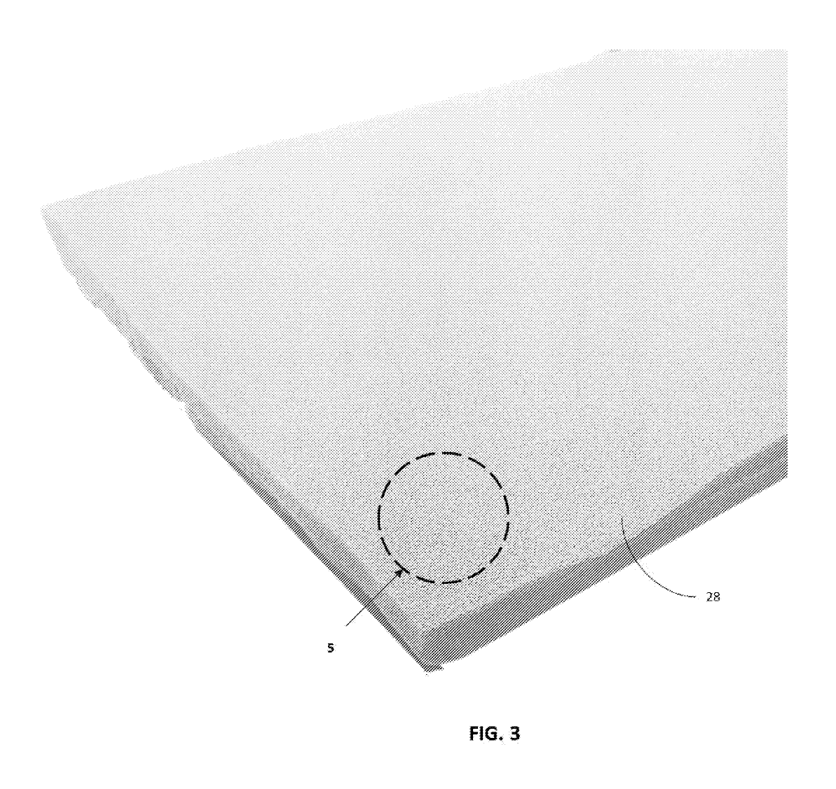

In one embodiment of the present invention, the substrate 28 is comprised of a high-surface-area material. By way of example and not of limitation, a 25 ppi (“pores per inch”) reticulated polyethylene foam, as shown in FIG. 3, has been used for the substrate 28 in the illustrated embodiment. Such a foam is commercially available from FOAM MART (Burbank, Calif.). Reticulated foam may be particularly effective in the present invention due to its porous, open cell structure, which imparts a high surface-area-to-volume ratio to the foam. Accordingly, for a given volume of the cavity 26 in the container 22, a reticulated foam provides an expansive surface area for treatment with the organo-functional silane-based molecules (indicated at 30 in FIG. 5), while minimally impacting the overall volume available for the fluid 27. Moreover, the high surface-area-to-volume ratio of reticulated foam allows for increased contact between the fluid 27 and, more particularly, the biological contaminants 38 within the fluid 27, and the organo-functional silane-based molecules 30.

While the present invention may be accomplished using a 25 ppi polyethylene foam as the substrate 28, as noted above, it is contemplated that any number of alternative materials may be used. By way of example, and not of limitation, it is contemplated that a wide variety of foams, including foams from materials other than polylethylene, may serve as the substrate 28. Moreover, those skilled in the art will recognize from the benefit of this disclosure that foams from a range of pores per inch (“ppi”) may be used, depending on the desired surface-area-to-volume ratio of the substrate 28. Additionally, the high-surface area substrate 28 need not be foam at all. Rather, it is contemplated that the substrate 28 may be comprised of a metallic, polymeric, fibrous, natural, or other material so long as the substrate 28 is capable of bonding with an organo-functional silane-based molecule, as discussed below, and is compatible with the fluid 27 to be biologically decontaminated.

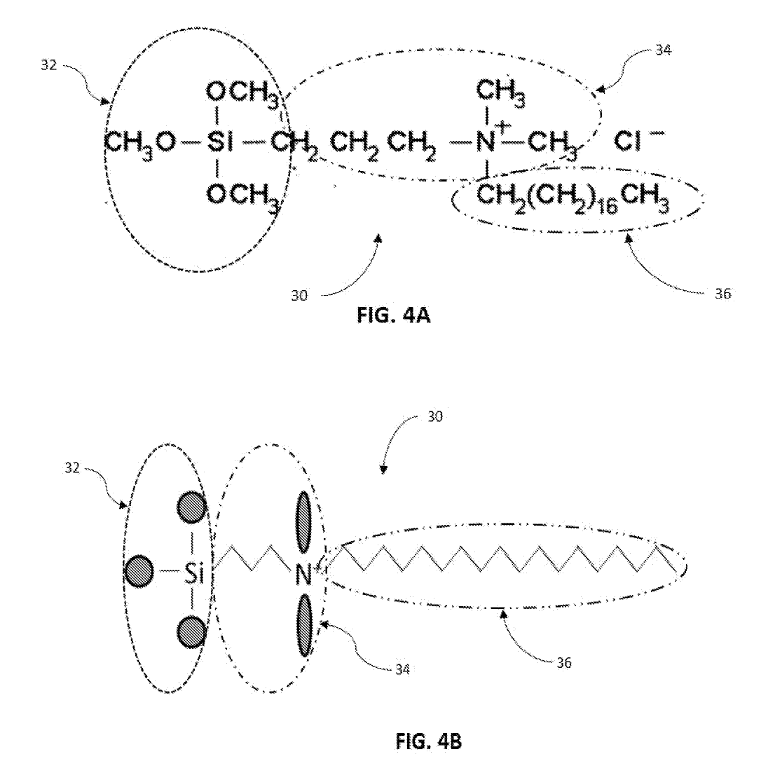

Turning now FIGS. 4A and 4B, a representative organo-functional silane-based molecule 30 comprising the decontamination solution is shown. In one embodiment of the present invention, and as shown in FIG. 4A, the organo-functional silane-based molecule 30 is 3-(trihydroxysilyl) propyldimethyloctadecyl ammonium chloride. The organo-functional molecule 30 includes a silane base, shown generally at 32, that is capable of covalently bonding with the surface 29 of the substrate 28 (see, e.g., FIG. 5). The silane base 32 of the organo-functional molecule 30 is bonded to a positively charged nitrogen group, shown generally at 34, which is operative to attract a biological contaminant 38 with a negatively charged cell membrane (as indicated at 40 in FIGS. 6A and 6B). The nitrogen group 34 of the organo-functional silane-based molecule 30 further is bonded to a long hydrocarbon chain, shown generally at 36, the hydrocarbon chain 36 operative to pierce the cell membrane 40 of the biological contaminant 38, as will be discussed. Such an exemplary molecule, as well as solutions comprising the same, are disclosed in U.S. Patent Application Publication No. US 2013/0017242, the disclosure of which is incorporated herein by reference in its entirety. Suitable solutions consisting essentially of organo-functional silane-based ammonium salts are commercially available under the name MONOFOIL (Coeus Technology, Anderson, Ind.). Still other commercially available solutions that may be used include those sold under the names AEGIS 444 RTU (Aegis Environments, Hunterville, N.C.), SISAM500 (SiShield Technologies, Inc., Atlanta, Ga.), BIOSHIELD 75 (IndusCo, Greensboro, N.C.), VSC 7200 (Vitec, Portsmouth, United Kingdom), ZTREX 72 (Piedmont Chemical, High Point, N.C.), SPORTSAIDE (Coating Specialist Group, Rovhester Hills, Mich.), MICROBEGUARD (Microbeguard, Elk Grove Village, Ill.), GOLDSHIELD 5 (AP Goldshield, Locust Valley, N.Y.), HM 4100 RTU (BioSafe, Inc., Pittsburgh, Pa.), MARQUAT 72 (W.M. Barr & Co., Memphis, Tenn.), M-PALE (DevMar Products, Nashville, Tenn.), SOONOCIDE (Coating Systems Laboratories, Chandler, Ariz.).

As discussed, the organo-functional silane-based molecule 30 shown in FIG. 4A is 3-(trihydroxysilyl) propyldimethyloctadecyl ammonium chloride. Those skilled in the art, however, will appreciate through the benefit of this disclosure that other organo-functional silane-based molecules may be used to accomplish the present invention. For instance, and without limitation, the organo-functional silane-based quarternary ammonium salt may be selected from the group consisting of 3-(trihydroxysilyl) propyldimeythylloctadecyl ammonium chloride, octadecyldimethyl trimethoxysilylpropyl ammonium chloride, and combinations thereof. For the purposes of the present invention, the only necessary characteristic of the chosen organo-functional silane-based molecule 30 is that it be capable of bonding to the substrate 28 and effectively eliminating, or otherwise rendering harmless to humans, the biological contaminant 38.

FIG. 4B depicts a simplified, stylized version of the organo-functional silane-based molecule shown in FIG. 4A.

In one embodiment, the decontamination solution includes a suspension of an organo-functional silane-based quartenary ammonium salt dissolved in a non-alcoholic solvent including water. The solution may be part of a liquid composition that includes a phenol ethoxylate, or part of a liquid composition that includes a phosphate ester. The concentration of the organo-functional silane-based quarternary ammonium salt is, in one embodiment, between approximately 0.10 percent to approximately 3.7 percent in water. In another embodiment, it is between approximately 1.0 percent and approximately 1.7 percent. In yet another embodiment, the concentration of the organo-functional silane-based quarternary ammonium salt is between approximately 1.3 percent and approximately 1.4 percent.

Turning now to FIG. 5, an enlarged view of the area circumscribed at 5 in FIG. 3 is shown, and illustrates, in stylized form, a portion of the surface 29 of the substrate 28. The substrate 28 has been treated with the solution consisting essentially of an organo-functional silane-based quarternary ammonium salt (so that the organo-functional silane-based molecules 30 are covalently bonded to the surface 29). Prefreably, though not necessarily, substantially all of the surface 29 of the substrate 28 is treated with the solution consisting essentially of an organo-functional silane-based quarternary ammonium salt.

During the treatment of the substrate with the aforementioned solution consisting essentially of an organo-functional silane-based quaternary ammonium salt, it has been found that the substrate will uptake a mass of solution that is greater than or equal to approximately 1% of the mass of the substrate, but less than or equal to approximately 20% of the mass of the substrate. Of course, those skilled in the art will understand that the exact mass ratio of solution to substrate will depend on the porosity of the substrate being treated.

As discussed above, in one exemplary embodiment of the present invention, a 25 ppi reticulated foam was used as the substrate 28. Reticulated foam provides an expansive surface area for the bonding of the organo-functional silane-based molecules 30. Accordingly, a relatively large number of organo-functional silane-based molecules 38 are available per unit volume to neutralize biological contaminants, as will be discussed.

It is contemplated that the particular substrate 28 discussed above may decontaminate different volumes of fluid 27. Moreover, it is contemplated that different substrates 28, as discussed above, and/or different substrate shapes and sizes, as discussed below, may be used in accordance with the present invention.

Referring next to FIGS. 6A and 6B, a biological contaminant 38 in the fluid 27 may draw near to the substrate 28 with its surface 29 treated with the at least one organo-functional silane-based molecule 30. As the biological contaminant 38 approaches the surface 29, the positively charged nitrogen group 34 attracts the negatively charged cell membrane 40 of the biological contaminant 38. Accordingly, the biological contaminant 38 is drawn toward the surface 29 and into the hydrocarbon chain 36. The hydrocarbon chain 36 then contacts, pierces and ruptures the cell membrane 40 of the biological contaminant 38. As the cell membrane 40 ruptures, a cytoplasm 42 effluxes from the biological contaminant 38, thus killing the biological contaminant 38. In this action, the at least one organo-functional silane-based molecule 30 is undisturbed and remains available for mechanically rupturing the cell membrane 40 of another biological contaminant 38.

Referring now to FIGS. 1, 2, 3, 4A, 4B, 5, 6A and 6B, the fluid purification system is employed as follows, according to the exemplary embodiment:

First, there is provided a substrate 28 at least a substantial area of the surface 29 of which is coated with the solution consisting essentially of an organo-functional silane-based quarternary ammonium salt at a concentration of between approximately 0.10 percent to approximately 3.7 percent in water.

At the time of the substrate's employment in the method of the present invention, the solution is, moreover, at least substantially dried on the substrate 28. In one embodiment, substantially drying of the solution on said substrate is accomplished through the evaporation of the water from said solution at room temperature. In other embodiments, alternatively, the step of drying is carried out under ambient conditions, under the controlled flow of air, under vacuum, under an inert gas (e.g., Nitrogen) environment. Drying may be carried out at any temperature suitable to the drying method; provided that the drying temperature is lower than the melting temperature of the open-cell foam substrate.

Second, the coated substrate 28 is placed in the container 22 so that the substrate 28 is substantially disposed within the cavity 26.

Next, a biologically contaminated fluid (such as water, by way of example and not of limitation) is introduced into the container 22. While in the container 22, the fluid 27 surrounds the substrate 28 and contacts the surface 29 treated with the at least one organo-functional silane-based molecule 30. In this manner, the biological contaminants 38 in the fluid 27 come into contact with the surface 29 of the substrate 28 treated with the at least one organo-functional silane-based molecule 30.

Next, the container 22 is agitated for a period of time sufficient to substantially biologically decontaminate the fluid therein. This promotes contact of the biological contaminants 38 with the surface 29 of the substrate 28. As the biological contaminants 38 contact the surface 29 treated with the at least one organo-functional silane-based molecule 30, their cell membranes 40 are pierced and ruptured by the organo-functional silane-based molecules 30 thus killing the biological contaminant 40, as described above.

Next, the container 22 is agitated for a period of time sufficient to substantially biologically decontaminate the fluid therein. This promotes contact of the biological contaminants 38 with the surface 29 of the substrate 28. As the biological contaminants 38 contact the surface 29 treated with the solution consisting essentially of an organo-functional silane-based quaternary ammonium salt, their cell membranes 40 are pierced and ruptured by the organo-functional silane-based molecules 30 thus killing the biological contaminant 40, as described above.

With or without agitation, the fluid being decontaminated is left in contact with the substrate for a period of time sufficient to effect the desired decontamination.

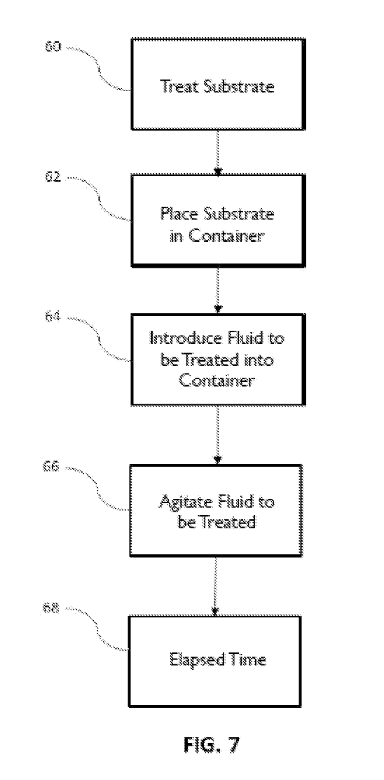

The foregoing methodology is illustrated in FIG. 7, wherein the above-described treatment (60), placement (62), fluid introduction (64), agitation (66) and time (68) steps are each depicted. Those skilled in the art will recognize from the benefit of this disclosure that the steps of the method of this invention need not be accomplished in the specific order described hereinabove. For example, it is contemplated that the substrate 28 may be treated with the solution consisting essentially of an organo-functional silane-based quaternary ammonium salt after insertion into the container 22, as well as before insertion, as shown in FIG. 7. Moreover, the fluid 27 may be introduced into the cavity 26 of the container 22 before the substrate 28 is inserted, as well as after, as depicted in FIG. 7. Additionally, the agitation step, as shown in box 66 may be accomplished after, or concurrently with, the time elapse step depicted in box 68.

Those skilled in the art will further understand from this disclosure that the agitation step 66 need not be conducted at all. Rather, the biological contaminants 38 may be brought into contact with the surface 29 of the substrate 28 through processes other than agitation. Such processes may include, for example, forced flow through the substrate 28, natural flow through the substrate 28, bulk splash of the fluid 27 in the container 22, convection, or natural movement of the biological contaminants 38.

In accordance with the present invention, the organo-functional silane-based molecules 38 are generally undisturbed by the mechanical rupturing of the cell membrane 40 of the biological contaminant 38. Rather, the organo-functional silane-based molecules 38 are available to rupture the cell membrane 40 of numerous biological contaminants 38. Accordingly, the fluid purification system 20 employed in the method of the present invention has a generally long useful life that is limited by, among other things, the frequency and intensity of use of the fluid purification system 20, the pH of the fluids 27 being purified, and the concentration of organo-functional silane-based molecules 38 on the surface 29 of the substrate 28.

In one embodiment of the present invention, and as shown in FIG. 2, the substrate 28 is completely disposed within the cavity 26 of the container 22. Of course, those skilled in the art will appreciate through the benefit of this disclosure that the substrate 28 need not be completely disposed within the cavity 26. Rather, for the purpose of the present invention, the container 22 must be capable of retaining the fluid 27 in contact with the substrate 28, and, more particularly, in contact with the surface 29 treated with the solution consisting essentially of an organo-functional silane-based quaternary ammonium salt. Accordingly, it is contemplated that, in some embodiments of the present disclosure, the container 22 may be open and further that the substrate 28 may protrude from the container 22. Thus, it is contemplated that the fluid purification system 20 of the present disclosure may be used to decontaminate fluids in a closed environment, such as in a bottle or tank, as well as in an open environment such as in a reservoir.

The substrate 28 shown in FIGS. 2 and 3, and the container 22 shown in FIGS. 1 and 2, are depicted as having a rectangular form. It is contemplated, however, that the container 22 and the substrate 28 may take any form desirable and suitable for the fluid 27 to be purified. Moreover, the substrate 28 may take any shape suitably adapted to the container 22 or, more particularly, to the cavity 26 within the container 22.

In accordance with the foregoing, those skilled in the art will now understand that the fluid purification method of the present disclosure may be advantageously used to decontaminate a vast range of fluids under a wide variety of conditions. By way of example and not limitation, backpackers and hikers may use the present invention to conveniently decontaminate water in canteens or hydration backpacks. Water treatment facilities may use the present invention to decontaminate water in reservoirs. Further, the present invention may be particularly well suited to decontaminate a wide range industrial fluids, including metal working fluids and coolants in their respective tanks, machines, or lines. Additionally, the invention may be advantageous in removing biological contaminants from blood and blood plasma. Moreover, the present invention may be used as part of a fluid stabilization system, wherein the conventional foam used for dampening the fore and aft motion of a fluid may be replaced by the substrate 28 of the present invention, such that the stabilized fluid may be purified of biological contaminants.

### Experimental Example 1

In an experimental example of the present inventive method, a 1′ (L)×1′ (W)×2″ thick substrate 28 comprised of 25 ppi reticulated polyethylene foam substrate was used to decontaminate a gallon of contaminated drinking water 27 contained in a one-gallon flask. More specifically, the foam substrate was at least substantially coated with a solution consisting essentially of an organo-functional silane-based quarternary ammonium salt at a concentration of between approximately 0.10 percent to approximately 3.7 percent in water. For the size substrate employed, from 2 to 4 ounces of the solution was sufficient to substantially coat the foam. After the solution had dried on the substrate, the substrate was introduced into the interior volume of the flask. As a flexible material, the foam substrate substantially conformed to, and substantially filled, the interior volume of the substrate. Thereafter, approximately one gallon of biologically contaminated drinking water was introduced into the interior volume of the flask, whereupon substantially all of the water was taken up by the porous foam. The flask was then agitated for approximately 20 minutes, whereafter the water was sufficiently decontaminated by contact between the biological contaminants in the water and the organo-functional silane-based quarternary ammonium salt molecules of the decontamination solution as to render the water safe for human consumption.

### Experimental Example 2

In another experimental example of the present inventive method, a substrate comprised of reticulated polyurethane foam substrate (melting point approximately 350°-375° F./density approximately 0.5-40 lbsₘ/ft³) (commercially available from FOAMEX INTERNATIONAL, INC., Linwood, Pa.) was at least substantially coated with a solution consisting essentially of an organo-functional silane-based quarternary ammonium salt at a concentration of between approximately 0.10 percent to approximately 3.7 percent in water (more specifically according to the example, the solution was the commercially available MONOFOIL (Coeus Technology, Anderson, Ind.)). After the solution was allowed to dry, the foam substrate was cut into multiple samples, each weighing about 0.50 g and dimensioned to fit into a 15 mL test tube. A further identical foam substrate, serving as a control, was not treated with the aforesaid solution and an identically-sized sample was cut from that untreated foam.

In a first round of this experimental example, each of a plurality of treated foam samples, designated A, C and D, were placed in a 15 mL test tube with 12 mL of PBS buffer solution (pH 7). The untreated foam sample, designated B, was likewise placed in a 15 mL test tube with 12 mL of the same PBS buffer solution. All foam sample-containing tubes A through D were shaken for approximately 60 minutes; then the buffer was removed from each tube. A further 12 mL of buffer was then introduced to each test tube A through D containing the foam substrate samples. Thereafter, all test tubes were challenged with a culture of S. aureus (about 10⁴ to 10⁵ CFU/mL) and shaken for approximately 60 minutes. Following this further shaking, approximately 100 uL aliquots were removed from each test tube (i.e., both “rinse” and those containing the foam samples) and spread on nutrient agar plates. These plates were subsequently incubated for 24 hrs at 37 degrees C. and any bacteria colonies formed during that period were promptly counted.

In this and subsequent rounds of testing described below, a plate showing no bacteria colonies was identified as a “99.9% Reduction,” while a plate showing a matte of colonies was identified as “0% Reduction”. The results of this first round of testing are summarized in Table 1, below.

TABLE 1

| Sample ID | Colony Count | % Reduction |
|---|---|---|
| Sample A “Treated Foam” | 0 | 99.9% |
| Sample B “Untreated Foam” | 10⁴ | 0% |
| Sample C “Treated Foam” | 0 | 99.9% |
| Sample D “Treated Foam” | 0 | 99.9% |

A second round of testing was conducted which was in all material respects identical to round 1, described above; except that each foam sample-containing test tube was rinsed twice; that is, all foam substrate-containing test tubes A through D were shaken for approximately 60 minutes; then the buffer was removed from each tube. A further 12 mL of buffer was then introduced to each foam sample-containing test tube A through D and these test tubes were shaken again for approximately 60 minutes; then the buffer was removed from each tube. Thereafter, all test tubes were challenged with a culture of S. aureus as described above. The results of this second round of testing are summarized in Table 2, below.

TABLE 2

| Sample ID | Colony Count | % Reduction |
|---|---|---|
| Sample A “Treated Foam” | 0 | 99.9% |
| Sample B “Untreated Foam” | 10⁴ | 0% |
| Sample C “Treated Foam” | 0 | 99.9% |
| Sample D “Treated Foam” | 0 | 99.9% |

A third round of testing was conducted which was in all material respects identical to round 1, described above; except that each foam sample-containing test tube was rinsed six (6) times; that is, all foam sample-containing tubes A through D were shaken for approximately 60 minutes; then the buffer was removed from each tube. A further 12 mL of buffer was then introduced to each foam sample-containing test tube A through D and those test tubes were shaken again for approximately 60 minutes; then the buffer was removed from each tube A through D. A further 12 mL of buffer was then introduced to each foam sample-containing test tube A through D and those test tubes were shaken again for approximately 60 minutes; then the buffer was removed from each tube. A further 12 mL of buffer was then introduced to each foam sample-containing test tube A through D and those test tubes were shaken again for approximately 60 minutes; then the buffer was removed from each tube. A further 12 mL of buffer was then introduced to each foam sample-containing test tube A through D and those test tubes were shaken again for approximately 60 minutes; then the buffer was removed from each tube. Finally, a further 12 mL of buffer was then introduced to each foam sample-containing test tube A through D and those test tubes were shaken again for approximately 60 minutes; then the buffer was removed from each tube. Thereafter, those test tubes A through D containing the foam samples were challenged with a culture of S. aureus as described above. The results of this third round of testing are summarized in Table 3, below.

TABLE 3

| Sample ID | Colony Count | % Reduction |
|---|---|---|
| Sample A “Treated Foam” | 0 | 99.9% |
| Sample B “Untreated Foam” | 10⁴ | 0% |
| Sample C “Treated Foam” | 0 | 99.9% |
| Sample D “Treated Foam” | 0 | 99.9% |

As manifest by the above-described results, the foam samples treated with the organo-functional silane-based quarternary ammonium salt solution were persistently effective at decontaminating the biologically contaminated fluid samples.

The above description is of preferred embodiments. Various alterations and changes can be made without departing from the spirit and broader aspects of the invention as defined in the appended claims, which are to be interpreted in accordance with the principles of patent law including the doctrine of equivalents. Any references to claim elements in the singular, for example, using the articles “a,” “an,” “the,” or “said,” is not to be construed as limiting the element to the singular.

The invention in which an exclusive property or privilege is claimed is defined as follows:

1. A method for decontaminating a biologically contaminated fluid, comprising the steps of:
providing a substrate comprising an open-cell foam at least substantially coated with a solution consisting essentially of an organo-functional silane-based quarternary ammonium salt at a concentration of between approximately 0.10 percent to approximately 3.7 percent in water;
at least substantially drying said solution on said substrate;
placing said coated substrate in a container;
introducing into said container a fluid to be decontaminated; and
agitating the container for a period of time sufficient to substantially biologically decontaminate said fluid.

2. The method of claim 1, wherein said fluid is selected from the group consisting of biologically-contaminated drinking water, fuels, industrial fluids, blood, and blood plasma.

3. The method of claim 1, wherein the substrate is an open-cell foam characterized by 25 pores per inch.

4. The method of claim 3, wherein the open-cell foam is a reticulated polyethylene foam.

5. The method of claim 3, wherein the open-cell foam is a reticulated polyurethane foam.

6. The method of claim 1, wherein the organo-functional silane-based quarternary ammonium salt is selected from the group consisting of 3-(trihydroxysilyl) propyldimeythylloctadecyl ammonium chloride, octadecyldimethyl trimethoxysilylpropyl ammonium chloride, and combinations thereof.

7. The method of claim 1, wherein the solution includes a suspension of organo-functional silane-based quartenary ammonium salt dissolved in a non-alcoholic solvent including water.

8. The method of claim 1, wherein the solution is part of a liquid composition that includes a phenol ethoxylate.

9. The method of claim 1, wherein the solution is part of a liquid composition that includes a phosphate ester.

10. The method of claim 1, wherein said concentration of said organo-functional silane-based quarternary ammonium salt is between about 1.0 percent and 1.7 percent, inclusive.

11. The method of claim 1, wherein said concentration of said organo-functional silane-based quarternary ammonium salt is between about 1.3 percent and 1.4 percent, inclusive.

12. The method of claim 1, wherein said step of at least substantially drying said solution on said substrate is carried out using one or more techniques selected from the group consisting of: evaporation of the water from said solution at room temperature;
drying under ambient conditions; drying under the controlled flow of air; and drying under vacuum; drying in an inert gas environment.

13. The method of claim 1, wherein the mass of solution coated on the substrate is between approximately 1% and approximately 20% of the mass of the substrate.
